(19) United States
(12) Patent Application Publication
(10) Pub. No.: US 2010/0318129 A1
(43) Pub. Date: Dec. 16, 2010

(54) DEFORMITY ALIGNMENT SYSTEM WITH REACTIVE FORCE BALANCING

(75) Inventors: Steven J. Seme, Savage, MN (US); Thomas J. Gisel, Chaska, MN (US)

Correspondence Address: FAEGRE & BENSON LLP;PATENT DOCKETING - INTELLECTUAL PROPERTY (, 2200 WELLS FARGO CENTER, 90 SOUTH SEVENTH STREET, MINNEAPOLIS, MN, 55402-3901, US

(73) Assignee: KSPINE, INC., Minnetonka, MN (US)

(21) Appl. No.: 12/485,796

(22) Filed: Jun. 16, 2009

(51) Int. Cl. A61B 17/70; A61B 17/88

(52) U.S. Cl. 606/254; 606/246; 606/278; 606/279

(57) ABSTRACT

Systems and methods for controlling a rotational effect on stabilizing vertebrae during and/or after deformity correction by directing reactive forces toward, on opposite sides of, and/or relatively closer to the transverse centers of rotation of the stabilizing vertebrae.

### BACKGROUND

Many systems have been designed to treat spinal deformities such as scoliosis, spondylolisthesis, and a variety of others. Primary surgical methods for correcting a spinal deformity utilize instrumentation to correct the deformity as much as possible, in combination with implantable hardware systems to rigidly stabilize and maintain the maximum achievable correction during a singular surgical intervention. At present, many of these implantable hardware systems rigidly fix the spinal column or allow limited growth and/or other movement of the spinal column, to help facilitate fusion after the spinal column has been moved to a final corrected position.

### SUMMARY

In some embodiments a system for correcting spinal deformities provides lateral translational corrective force(s) and/or derotational corrective force(s) on a spinal column tending to exhibit a defective curvature. Some embodiments relate to controlling a rotational effect on stabilizing vertebrae during deformity correction by directing reactive forces toward the transverse centers of rotation of the stabilizing vertebrae, for example. By balancing such reactive forces, vertebral derotation in a desired, target region of the spinal column is encouraged while rotational moments on stabilizing vertebrae to which the system is secured are reduced.

Some embodiments relate to a system for correction of a spinal column having a target region exhibiting a spinal deformity, the system including a stabilizing member and first and second stabilizing anchors on stabilizing vertebrae and the system being configured such that upon tensioning a connector to a correction vertebra a corrective force is exerted on the correction vertebra and a reactive force is exerted on first and second stabilizing vertebrae such that the corrective force passes at an offset distance from a transverse center of rotation of the correction vertebra and the reactive force passes on an opposite side of the transverse centers of rotation and/or relatively closer to the transverse centers of rotation of each of the first and second stabilizing vertebrae, such that the system helps correct the deformed target region.

This summary is not meant to be limiting in nature. While multiple embodiments are disclosed herein, still other embodiments of the present invention will become apparent to those skilled in the art from the following detailed description, which shows and describes illustrative embodiments of the invention. Accordingly, the drawings and detailed description are to be regarded as illustrative in nature and not restrictive.

### BRIEF DESCRIPTION OF THE DRAWINGS

FIG. 1 shows an exemplary system for correcting a spinal deformity, according to some embodiments.

FIG. 2 shows a correction anchor of the system of FIG. 1, according to some embodiments.

FIGS. 3 and 4 show a tensioner of the system of FIG. 1, according to some embodiments.

FIG. 5 is a diagrammatical representation of another system, according to some embodiments.

FIGS. 6 and 7 are transverse plane views of portions of the system of FIG. 5, according to some embodiments.

FIG. 8 is a transverse plane view showing the portions of FIGS. 6 and 7 overlaid onto one another with a vertebra in a first, uncorrected position, according to some embodiments.

FIG. 9 is a transverse plane view showing the vertebra of FIG. 8 in a second, corrected position, according to some embodiments.

FIG. 10 is a transverse plane view of the system of FIG. 5 showing various features used for pre-selecting system configuration, according to some embodiments.

Various embodiments have been shown by way of example in the drawings and are described in detail below. As stated above, the intention, however, is not to limit the invention by providing such examples.

### DETAILED DESCRIPTION

Some embodiments relate to a system for correcting spinal deformities, as well as associated methods and devices. In general terms, the system provides lateral translational corrective force(s) and/or derotational corrective force(s) on a spinal column tending to exhibit a defective curvature. Some features of the system include highly adaptive hardware for effective application of derotational corrective force(s) by balancing reactive forces resulting from corrective forces imposed on the spinal column in a target defective region. By balancing such reactive forces, vertebral body translation and/or derotation in a desired, target region of the spinal column is encouraged while rotational moments on stabilizing vertebrae to which the system is secured are reduced. In some embodiments, the system facilitates incremental correction, gross correction, and/or correction maintenance as desired.

Various planes and associated directions are referenced in the following description, including a sagittal plane defined by two axes, one drawn between a head (superior) and tail (inferior) of the body and one drawn between a back (posterior) and front (anterior) of the body; a coronal plane defined by two axes, one drawn between a center (medial) to side (lateral) of the body and one drawn between a head (superior) and tail (inferior) of the body; and a transverse plane defined by two axes, one drawn between a back and front of the body and one drawn between a center and side of the body.

Also, the terms pitch, roll, and yaw are used, where roll generally refers to angulation, or rotation, in a first plane through which a longitudinal axis of a body orthogonally passes (e.g., rotation about a longitudinal axis corresponding to the spinal column), pitch refers to angulation, or rotation, in a second plane orthogonal to the first plane, and yaw refers to angulation, or rotation, in a third plane orthogonal to the first and second planes. In some embodiments, pitch is angulation in the sagittal plane, yaw is angulation in the coronal plane, and roll is angulation in the transverse plane. In various embodiments, changes in pitch, yaw, and/or roll occur concurrently or separately as desired. Moreover, as used herein, “lateral translation” is not limited to translation along the medial-lateral axis (in either the lateral-medial or medial-lateral direction(s)) unless specified as such.

The term “Cobb's angle,” or “Cobb angle,” is an angular measurement used to evaluate a degree of spinal deformity, for example in association with scoliosis. The evaluation, typically performed on an anterior-posterior radiographic projection of the spine, includes identifying an apex of the deformity corresponding to an apical vertebra (e.g., the most laterally displaced and rotated vertebra with the least tilted end plate) as well as the transitional vertebrae at the upper and lower ends of the target defect area, or region. Typically, the transitional vertebrae are the most superior and inferior vertebrae closest to the deformity which are least displaced and rotated and have maximally tilted end plates. A line is drawn along the superior end plate of the superior end vertebra and a second line is drawn along the inferior end plate of the inferior end vertebra. The line(s) can also be drawn through the pedicles. An angle between these two lines (or lines drawn perpendicular to them) is measured as the Cobb angle. As a general rule, a Cobb angle of 10 degrees is regarded as a minimum angulation to define scoliosis.

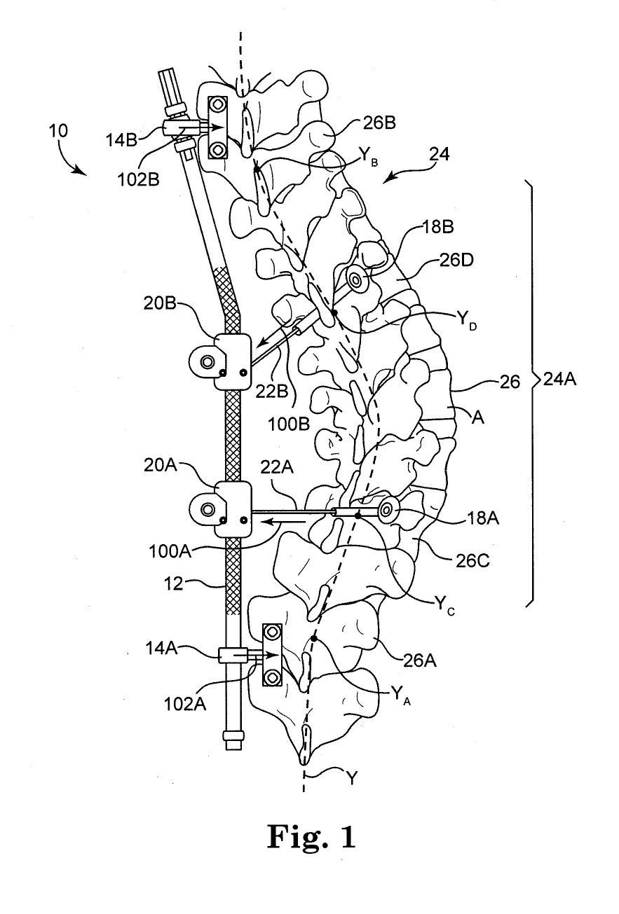

FIG. 1 is a perspective view of a system 10 for correcting a spine tending to exhibit a spinal deformity, according to some embodiments. As shown in FIG. 1, the system 10 includes a stabilizing member 12, a plurality of stabilizing anchors 14, including a first stabilizing anchor 14A and a second stabilizing anchor 14B, a plurality of correction anchors 18 including a first correction anchor 18A and a second correction anchor 18B, a plurality of tensioners 20 including a first tensioner 20A and a second tensioner 20B, and a plurality of connectors 22 including a first connector 22A and a second connector 22B. As shown, the system 10 is secured to a spinal column 24 formed of a plurality of vertebrae 26, including a first vertebra 26A, a second vertebra 26B, a third vertebra 26C, and a fourth vertebra 26D.

As shown, the spinal column 24 has a transverse centerline of rotation Y, also described as a longitudinal axis of rotation. In some embodiments, the transverse centerline rotation Y of the spinal column 24 generally corresponds to a mid-distance position of the spinal canal (not shown) extending through the spinal column 24, where each vertebra 26 has a transverse center of rotation generally located on the transverse centerline of rotation Y. For example, as shown in FIG. 1 each of the first, second, third, and fourth vertebrae 26A, 26B, 26C, 26D has a transverse center of rotation Y_{A}, Y_{B}, Y_{C}, Y_{D}, respectively, along the transverse centerline of rotation Y.

In some embodiments, the stabilizing member 12 is also referred to as a rod or alignment member; the stabilizing anchors 14 are also referred to as alignment supports or guides; the correction anchors 18 are also referred to as anchor arms or vertebral levers, the tensioners 20 are also referred to as adjustment mechanisms or tying devices, and the connectors 22 are also referred to as force directing members or cables, for example.

Examples of suitable stabilizing members 12, stabilizing anchors 14, correction anchors 18, tensioners 20, and connectors 22 according to some embodiments are described in U.S. application Ser. No. 12/411,562, filed Mar. 26, 2009, and entitled “Semi-Constrained Anchoring System”; U.S. application Ser. No. 11/196,952, filed Aug. 3, 2005, and entitled “Device and Method for Correcting a Spinal Deformity”; and U.S. application Ser. No. 12/134,058, filed Jun. 5, 2008, and entitled “Medical Device and Method to Correct Deformity,” the entire contents of each which are incorporated herein by reference.

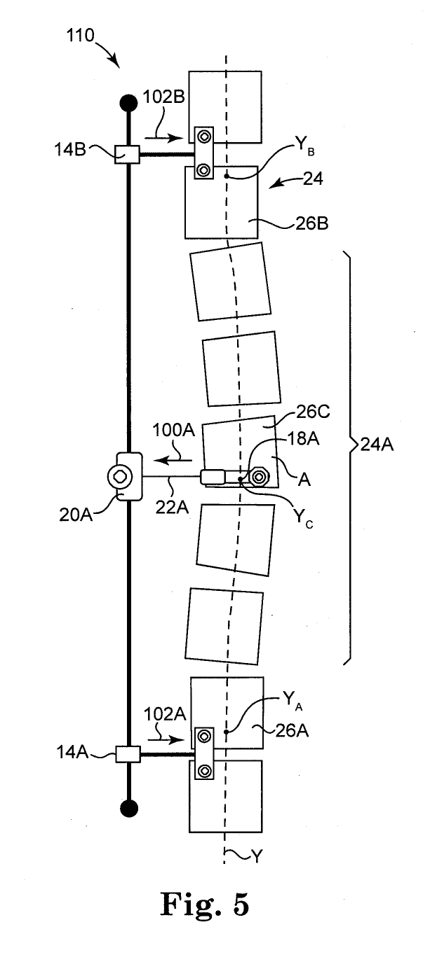

Although the system 10 is shown with two stabilizing anchors 14, two correction anchors 18, two tensioners 20, and two connectors 22, a greater or fewer number thereof are implemented as appropriate. For example, as shown in FIG. 5, in some embodiments a single one of the correction anchors 18 is secured to one of the vertebrae 26 near an apex A of defective curvature with one of the corresponding connectors 22 and tensioners 20 being coupled to the corresponding correction anchor 18.

As shown in FIG. 1, the first and second correction anchors 18A, 18B are fixed to a target region 24A of the spinal column 24 tending to exhibit an abnormal, or defective curvature (e.g., scoliosis) in need of correction. The system 10 is optionally used to apply derotational and/or lateral translational forces on the target region 24A of the spinal column 24 to translate and/or maintain the spinal column 24 at a desired curvature.

As described in greater detail, the system 10 is adapted to apply lateral translational and derotational forces during various stages of deformity correction and/or correction maintenance in a manner that helps minimize rotational effects from complementary reactive forces on the spinal column 24. Restated, in some embodiments the system 10 is adapted to derotate the vertebrae 26 in the target region 24A while minimizing derotational forces on a remainder of the spinal column 24 during and/or after correction of a spinal defect. In some embodiments, incremental adjustments are made to the system 10 to achieve a desired correction. In others, a single, or gross adjustment is made to the system 10 to correct to a desired curvature. In still other embodiments, the target region 24A of the spinal column 24 is adjusted to a more natural or desired curvature using other, non-implanted hardware, prior to or in conjunction with implanting and securing the system 10 to the spinal column 24.

FIG. 1 shows the stabilizing member 12 having a bend according to some embodiments, although the stabilizing member 12 is straight or substantially straight in other embodiments. In FIG. 1, the bend in the stabilizing member 12 is generally shown for illustrative purposes, where the stabilizing member 12 is optionally bent in the sagittal and/or coronal planes. In some embodiments, the stabilizing member 12 is contoured to a desired curvature of the spinal column 24, where the stabilizing member can be contoured to an expected shape of the spinal column 24 at any of a variety of stages of correction (e.g., from partially corrected to fully corrected). The stabilizing member 12 is optionally formed of a variety of materials, including stainless steel, titanium, suitable polymeric materials such as PEEK, superelastic materials, such as a shape memory materials, or others. Some examples of suitable stabilizing members are provided in U.S. application Ser. No. 12/411,558 filed on Mar. 26, 2009 and entitled, “Alignment System with Longitudinal Support Features,” the entire contents of which are incorporated herein by reference.

In some embodiments, the stabilizing member 12 is substantially elongate and rigid, defining a substantially round cross-section with a mean diameter of about 6 mm and being formed of a suitable biocompatible material, such as titanium alloy ASTM F136. If desired, the stabilizing member 12 incorporates some flex, or springiness while substantially rigidly retaining its shape. The cross-sectional shape of the stabilizing member 12, including various portions thereof, is not limited to circular cross-sections and varies lengthwise in cross-section as desired. As will be described in greater detail, the stabilizing member 12 is adapted, or otherwise structured, to extend along the spinal column 24 at a desired spacing from the vertebrae 26 of the spinal column 24 where, in some embodiments, the stabilizing member 12 is partially or fully contoured to a typical, corrected curvature of the spinal column 24.

The stabilizing member 12 has a longitudinal axis X and where the stabilizing member 12 is substantially straight, the longitudinal axis X is substantially straight. Where the stabilizing member 12 is substantially curved or angled, the longitudinal axis X is similarly curved or angled. The stabilizing member 12 is optionally continuously formed or as separate, connected parts as desired. The stabilizing member 12 also optionally includes features for adjusting a length of the stabilizing member 12 as desired.

FIG. 1 shows the pair of stabilizing anchors 14A, 14B which are adapted, or otherwise structured, to be mounted or fixed to one or more stabilizing vertebrae, such as the first and second vertebrae 26A, 26B. The first and second stabilizing anchors 14A, 14B are further adapted to receive, and include means for receiving, the stabilizing member 12 such that the stabilizing member 12 is secured laterally, against lateral translation relative to the first and second stabilizing anchors 14A, 14B.

In some embodiments, the stabilizing anchors 14 are secured to a single one of the vertebra 26 (e.g., laterally across the vertebra at the pedicles, or at a single point, such as a single pedicle). The stabilizing anchors 14 are optionally adapted to be secured to multiple locations or a single location as desired. The first and second stabilizing anchors 14A, 14B are each secured to a single vertebra in some embodiments or multiple vertebrae in others, such as an additional, adjacent one of the vertebra 26. As shown in FIG. 1, the first and second stabilizing anchors 14A, 14B are secured to the first and second vertebrae 26A, 26B, respectively, as well as one of the vertebrae 26 adjacent each of the first and second vertebrae 26A, 26B. As one example, the first stabilizing anchor 14A is optionally secured to the pedicles of the L3-L4 vertebrae. As appropriate, the vertebrae 26 to which the stabilizing anchors 14 are secured are transitional vertebrae and/or vertebrae adjacent transitional vertebrae, where the vertebrae 26 to which the stabilizing anchors 14 are secured act as stabilizing vertebrae for the system 10.

As received by the first and second stabilizing anchors 14A, 14B, the stabilizing member 12 is semi-constrained by the stabilizing anchors 14, the stabilizing member 12 being free to move with natural movements of the spinal column 24 while being substantially prevented from translating in a direction that is substantially perpendicular to the longitudinal axis X of the stabilizing member 12 at each of the stabilizing anchors 14A, 14B. Some suitable examples of semi-constrained anchoring systems are described in the previously-incorporated application entitled “Semi-Constrained Anchoring System.”

In some embodiments, the stabilizing member 12 is able to slide axially, or translate axially, along the longitudinal axis X, relative to the first and/or second stabilizing anchors 14A, 14B. The stabilizing member 12 is able to slide and to change in at least pitch and yaw at the first and second stabilizing anchors 14A, 14B. For example, the stabilizing member 12 is also able to change in roll at the first and/or the second stabilizing anchors 14A, 14B according to some embodiments. In various embodiments, the stabilizing anchors 14A, 14B limit the degrees of freedom of the stabilizing member 12 within a desired range of movement as desired.

Thus, in some embodiments, the stabilizing anchors 14 are adapted to receive the stabilizing member 12 and secure the stabilizing member 12 against substantial lateral translation relative to stabilizing vertebrae (e.g., the first and second vertebrae 26A, 26B). For example, the vertebrae 26A, 26B (as well as secondary vertebra to which the stabilizing anchors 14 are secured) are used to stabilize the stabilizing member 12 which defines a line of reference from which to adjust defective curvature by providing a series of anchor points toward which the target region 24A is able to be pulled.

The first and second correction anchors 18A, 18B are optionally substantially similar, and thus various features of both the first and second correction anchors 18A, 18B are described in association with the first correction anchor 18A. Features of the first correction anchor 18A are designated with reference numbers followed by an “A” and similar features of the second correction anchor 18B are designated with similar reference numbers followed by a “B.”

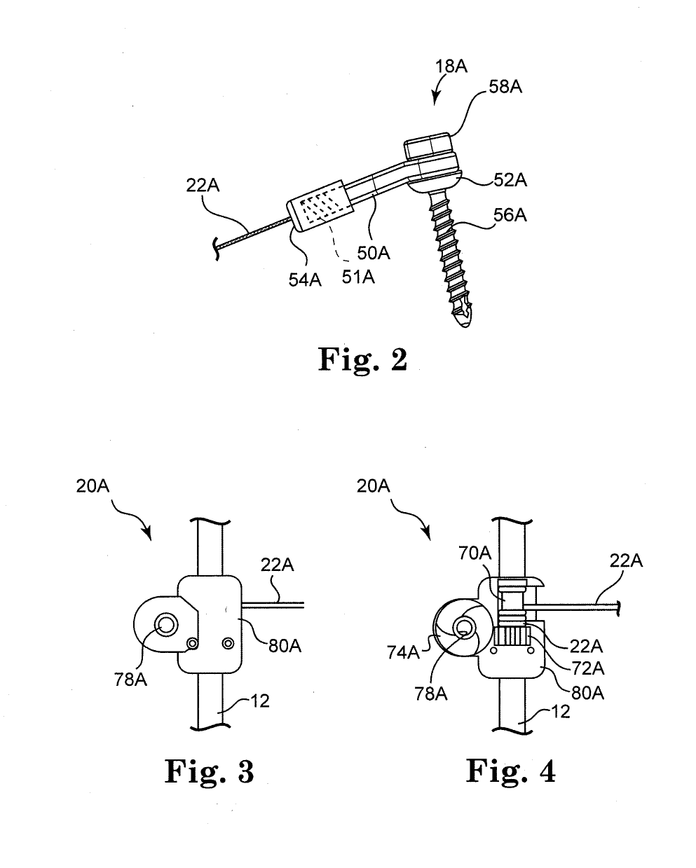

FIG. 2 shows the first correction anchor 18A according to some embodiments. As shown, the first correction anchor 18A is generally L-shaped, where the first correction anchor 18A includes an arm 50A with optional threading 51A (shown in broken lines) and a head 52A assembled to one another in a generally L-shaped configuration. The first correction anchor 18A is optionally substantially rigid. In some embodiments, the arm 50A extends from the head 52A to a terminal coupler 54A and is disposed generally perpendicular to the head 52A. In some embodiments, a length of the correction anchor 18A is adjustable using the threading 51A (e.g., by adjusting the location of the terminal coupler 54A on the threading 51A). If desired, the arm 50A includes a bend and/or extends at an angle from the head 52A. The arm 50A is optionally secured about, and rotatable relative to the head 52A and is adapted to extend across one of the vertebrae 26, for example, from one side of the spinal column 24 to an opposite side of the spinal column 24.

The first correction anchor 18A is secured to the third vertebra 26C such that the arm 50A extends across the third vertebra 26C either adjacent to the spinous processes or through a hole or hollowed portion in the spinous processes of the third vertebra 26C. In some embodiments, the third vertebra 26C is an apical vertebra at the apex A of the target region 24A (FIG. 1).

The head 52A of the correction anchor 18A is optionally adapted or otherwise structured to be fixed to a portion of the third vertebra 26C, such as a pedicle of the third vertebra 26C. The head 52A includes a body portion 56A and a cap portion 58A. The head 52A includes and/or is adapted to work in conjunction with any of a variety of means for securing to the third vertebra 26C. For example, the body portion 56A is optionally configured as a pedicle screw. Assembly of the first correction anchor 18A includes receiving the arm 50A on the body portion 56A of the head 52A and screwing or otherwise securing the cap portion 58A onto the body portion 56A. In some embodiments, the arm 50A is rotatable relative to the head 52A upon assembly of the correction anchor 18A.

The first tensioner 20A is shown in FIGS. 3 and 4, where FIG. 4 shows the first tensioner 20A with a portion removed to illustrate inner features thereof. The first and second tensioners 20A, 20B are optionally substantially similar, and thus various features of both the first and second tensioners 20A, 20B are described in association with the first tensioner 20A. Features of the first tensioner 20A are designated with reference numbers followed by an “A” and similar features of the second tensioner 20B are designated with similar reference numbers followed by a “B.”

Generally, the first tensioner 20A provides means for securing the first connector 22A to the stabilizing member 12. In some embodiments, the first tensioner 20A, also described as an adjustment mechanism or coupler, is further adapted to adjust, and provides means for adjusting the effective length of the first connector 22A.

In some embodiments, the first tensioner 20A includes a reel 70A, a circumferential gear 72A surrounding the reel 70A, a vertical gear 74A in contact with the circumferential gear 72A, an actuation head 78A, and a housing 80A.

The reel 70A, as well as the circumferential gear 72A and vertical gear 74A are maintained at least partially within the housing 80A. In turn, the housing 80A is adapted to be secured to the stabilizing member 12. For example, the housing 80A optionally forms a central lumen (not shown) through which the stabilizing member 12 is receivable. Upon inserting the stabilizing member 12 through the central lumen, the housing 80A is adapted to be clamped onto the stabilizing member 12. Some examples of suitable tensioners are described in U.S. application Ser. No. 12/134,058, filed on Jun. 5, 2008 and entitled, “Medical Device and Method to Correct Deformity,” the entire contents of which are incorporated herein by reference.

In some embodiments, the housing 80A incorporates a clamshell design (e.g., a first portion adjustably secured to a second portion) adapted to be tightened onto the stabilizing member 12 (e.g., using one or more fasteners). Thus, in some embodiments, the first tensioner 20A is substantially fixed with respect to the stabilizing member 12. In other embodiments, however, the first tensioner 20A is movable with respect to the stabilizing member 12, for example being able to rotate about the stabilizing member 12 and/or slide along the stabilizing member 12.

The first connector 22A is attached or secured to the reel 70A and passes out of the housing 80A through an appropriately sized opening in the housing 80A. Actuation of the vertical gear 74A via the actuation head 78A turns the circumferential gear 72A, which turns the reel 70A, thus winding (or unwinding, depending on the direction in which the reel 70A is turned) the first connector 22A about the reel 70A. Rotation of the reel 70A in the appropriate direction draws the first connector 22A in toward the first tensioner 20A, pulling the first correction anchor 18A (FIG. 1) toward the first tensioner 20A according to some methods of correcting a spinal defect.

From the foregoing, it should also be understood that the second connector 22B and the second tensioner 20B shown in FIG. 1 are similarly coupled, where actuation of the second tensioner 20B modifies an effective length of the second connector 22B, either drawing the second connector 22B toward the second tensioner 20B or letting out the second connector 22B away from the second tensioner 20B.

The connectors 22A, 22B are optionally substantially similar, and thus various features of the first and second connectors 22A, 22B are described in association with the first connector 22A. Features of the first connector 22A are designated with reference numbers followed by an “A” and similar features of the second connector 22B are designated with similar reference numbers followed by a “B.”

In some embodiments, the first connector 22A is substantially flexible such that the first connector 22A is able to be pivoted in multiple directions (e.g., to facilitate a polyaxial connection to the correction anchor 18A and/or the tensioner 22A). Such flexibility additionally or alternatively facilitates spooling or winding of the first connector 22A, for example. Suitable flexible materials for forming the first connector 22A include wire and stranded cables, monofilament polymer materials, multifilament polymer materials, multifilament carbon or ceramic fibers, and others. In some embodiments, the first connector 22A is formed of stainless steel or titanium wire or cable, although a variety of materials are contemplated.

As shown in FIG. 1, the first connector 22A, also described as a force directing member or a cable, is adapted to be secured to the first correction anchor 18A and the first tensioner 20A, the first connector 22A defining an effective length between the first tensioner 20A and the first correction anchor 18A, and thus the stabilizing member 12 (although, in some embodiments, the first connector 22A is secured directly to the stabilizing member 12). As described, in some embodiments, the first tensioner 20A is adapted to modify, and provides means for modifying, the effective length of the first connector 22A.

In view of the foregoing, assembly and use of the system 10 according to some embodiments generally includes attaching the stabilizing anchors 14 on superior and/or inferior locations of the target region 24A, for example to transitional vertebrae characterizing a scoliotic curvature of the spinal column 24. In some embodiments, the target region 24A includes those of the vertebrae 26 in need, or in greater need, of correction. In operation, the connectors 22 couple the correction anchors 18 to the stabilizing member 12 and, by retracting the connectors 22 toward the stabilizing member 12, the spinal column 24 is brought into more natural alignment.

The system 10 is optionally used for incremental correction, for gross correction, and/or for maintaining a correction as desired. For example, the connectors 22 are optionally retracted incrementally as part of one or more procedures using the tensioners 20. In other embodiments, a single, gross adjustment is made using the tensioners 20 or other device(s) to accomplish a desired correction. In still other embodiments, a correction is made using other hardware, prior to or in conjunction with securing the system 10 to the spinal column 24, where the system 10 is utilized to maintain the desired correction.

In some embodiments, assembly of the system 10 includes securing the first and second tensioners 20A, 20B to the stabilizing member 12, where FIG. 1 shows the system 10 in an assembled state. The first and second stabilizing anchors 14A, 14B are secured to the first and second vertebrae 26A, 26B, respectively (e.g., using pedicle screws). In some embodiments, the first and second vertebrae 26A, 26B are transitional vertebrae, are adjacent the transitional vertebrae, and/or are generally located posteriorly and anteriorly, proximate the upper and lower ends, of the target region 24A tending to exhibit defective curvature.

The stabilizing member 12 is received in the first and second stabilizing anchors 14A, 14B to secure the stabilizing member 12 against lateral translation relative to the spinal column 24, while still providing semi-constrained movement as desired. For example, as previously described, features of the first and second stabilizing anchors 14A, 14B are selected to permit changes in pitch, yaw, roll, and/or axial sliding of the stabilizing member 12.

In some embodiments, the first and second correction anchors 18A, 18B are secured to the third and fourth vertebrae 24C, 24D, respectively and the first and second connectors 22A, 22B are secured to the first and second correction anchors 18A, 18B and the first and second tensioners 20A, 20B, respectively. The first connector 22A is assembled to the first correction anchor 18A by securing the second end of the first connector 22A to the first correction anchor 18A proximate the terminal coupler 54A (FIG. 2) thereof.

In some embodiments, the first connector 22A is secured into the terminal coupler 54A of the first correction anchor 18A, and extends along at least a portion of the arm 50A to the head 52A (FIG. 2), although the first connector 22A is attached at any location along the arm 50A and/or the head 52A of the first correction anchor 18A as appropriate. In some embodiments, flexibility of the first connector 22A facilitates a polyaxial connection between the first connector 22A and the first correction anchor 18A at the terminal coupler 54A, the first connector 22A being able to flex, bend, or otherwise angulate freely at the terminal coupler 54A. The first connector 22A is securable to the first correction anchor 18A via a variety of methods, including welding, adhesives, tying, and/or screw fixation, for example.

The second connector 22B and the second correction anchor 18B are optionally secured or connected together using similar approaches.

In some embodiments, the first correction anchor 18A is secured through a pedicle of the third vertebra 26C on a convex side of a scoliotic deformity (i.e., the outwardly curving side of the deformity). The arm 50A of the first correction anchor 18A is optionally configured to pass in a trajectory that is substantially parallel to an endplate of the third vertebra 26C and/or parallel to disc space defined between the third vertebra 26C and an adjacent vertebra.

As shown in FIG. 1, the arm 50A extends across the third vertebra 26C, passing the transverse centerline of rotation Y of the spinal column 24, and in particular the transverse center of rotation Y_{C} of the third vertebrae 24C to the concave side of the deformity (i.e., the inwardly curving side of the deformity). As described in greater detail, by extending the arm 50A across the third vertebra 26C, the correction anchor 18A is able to be secured on one side of the spinal column 24 (e.g., to the pedicle on the side tending to exhibit convex curvature associated with scoliosis) while facilitating application of a force to the opposite side of the spinal column 24 (e.g., on the opposite side tending to exhibit concave curvature associated with scoliosis) to rotate the third vertebra 26C. In other embodiments, the correction anchors 18 are secured at other locations on the spinal column 24 (e.g., to the vertebral body boney anatomy on the concave side at or lateral to pedicle entrance location(s)).

In some embodiments, in a similar manner in which the first correction anchor 18A is secured to the third vertebra 26C, the second correction anchor 18B is secured to the fourth vertebra 26D.

Although some embodiment correction anchors have been described, a variety of additional or alternate correction anchor designs and features are also contemplated (e.g., one or bands around the transverse process, staples, clips, and others).

In order to achieve a desired correction, in some embodiments the stabilizing member 12 is secured to the spinal column 24 at a pre-selected offset from the transverse centerline of rotation Y. By securing the stabilizing member 12 against lateral translation at the first and second stabilizing anchors 14A, 14B, the stabilizing member 12 acts to provide a series of stabilizing points adjacent the spinal column 24 to which the connectors 22 and connection anchors 18 can be attached. In at least this manner, the connectors 22 are able to be pulled upon or tensioned from the stabilizing member 12 and associated anchor points in order to derotate and move the vertebrae 26 of the target region 24A (or “defect vertebrae”) toward the stabilizing member 12.

In some embodiments, the first tensioner 20A is optionally adapted to modify and provides means for modifying the effective length of the connector 22A and/or is adapted for securing and provides means for securing the first connector 22A to the stabilizing member 12. By shortening the effective length between the first tensioner 20A and the first correction anchor 18A, a corrective force 100 in the form of a tension is exerted on the third vertebra 26C and correction anchor 18A along the connector 22A, which is resisted by the stabilizing member 12. In turn, a reactive force 102 is exerted through the stabilizing member 12 into the first and second stabilizing anchors 14A, 14B and ultimately back into the spinal column 24 at the first and second vertebrae 26A, 26B. Thus, the first and second vertebrae 26A, 26B, as well as any other vertebrae 26 to which the first and second stabilizing anchors 14A, 14B are connected serve as stabilizing vertebrae for the system 10.

In other words, the corrective force 100 exerts a pull effect onto the spinal column 24 while the reactive force 102 applies a push effect onto the spinal column 24, allowing the spinal column 24 to be corrected to a more natural alignment between the stabilizing anchors 14. Where multiple connectors 22 and correction anchors 18 are employed, each of the connectors 22 is associated with a component of the corrective force 100. For example, the first connector 22A is optionally associated with a first component 100A of the corrective force 100 and the second connector 22B with a second component 100B of the corrective force 100. In some embodiments, the sum of the first and second components 100A, 100B equal the corrective force 100. Where the first connector 22A is the sole connector applying a corrective force, the first component 100A is equal to the corrective force 100. Where additional corrective anchors 18 and connectors 22 are in operation, additional components of the corrective force 100 would be indicated in such embodiments.

In turn, in some embodiments the first stabilizing anchor 14A corresponds to a first component 102A of the reactive force 102 and the second stabilizing anchor 14B corresponds to a second component 102B of the reactive force 102, the sum of the first and second components 102A, 102B equaling the reactive force 102.

In view of the foregoing, some methods of treating the tendency of the spinal column 24 to exhibit the defect, or deformity (e.g., scoliosis) include implanting the stabilizing anchors 14 on either side of an exhibited defect, attaching the stabilizing member 12 between the stabilizing anchors 14, implanting the correction anchors 18 onto at least one of the vertebrae 26 in the target region 24A tending to exhibit an undesirable rotational position, coupling the correction anchors 18 to the stabilizing member 12 using a polyaxial connection using the connectors 22, and tensioning the connectors 22 to correct the position of the at least one vertebra 26.

Although in some embodiments the system 10 is adapted to span the spinal deformity (e.g., being secured to stabilizing vertebrae outside the target region 24A), in other embodiments, the stabilizing vertebrae reside within the region of spinal deformity (e.g., reducing a number of vertebral levels between the cephalad and caudal instrumented levels). Moreover, while some embodiment systems have been shown with the stabilizing member 12, stabilizing anchors 14, and connectors 22 secured on a side of the spinal column tending to exhibit a concave curvature, other embodiments are contemplated in which such components are located on an opposite side of the spinal column (e.g., a side tending to exhibit convex curvature).

In some embodiments, the appropriate offset of the stabilizing member 12, and in particular, the offset at which each of the first and second stabilizing anchors 14 maintain the stabilizing member 12 (e.g., angular position of the stabilizing member 12 relative to the vertebrae 26 of the spinal column 24 as well as the transverse lateral distance from the spinal column 24), is determined experimentally and/or theoretically for a particular type of defect. For example, through radiological evaluations (i.e. x-rays, MRI or CT) a surgeon, computer, or other entity preoperatively characterize the deformity (Cobb angle, apical translation, apical rotation, apical pedicle-to-pedicle width, or other characteristic).

Based on such characterization data, theoretical location of the stabilizing member offset, resulting corrective forces, reactive forces and locations with respect to the center of rotation are determined based upon geometric modeling (e.g., similarly to the exemplary stabilizing member location determinations described above). The surgeon or other user is then able to select from a variety of differently sized and configured stabilizing anchors 18, stabilizing members 12, and/or correction anchors 14, for example, based upon the determined values. Thus, a surgeon or other user is able to select appropriately configured hardware of the system 10 based upon a preoperative characterization of the spinal column 24.

An alternative or additional manner of helping ensure appropriate system force balancing includes tensioning the connectors 22 (e.g., by making minor adjustments to the effective lengths of the connectors 22) while inspecting the stabilizing vertebrae and vertebrae in the target region 24A. Examination of the system 10 and the vertebrae 26 is accomplished by direct visual inspection, radiography, force sensors, accelerometers, or other measurement technique as appropriate.

In some embodiments, a patient is placed in an at rest position (e.g., laying belly down on a table) and, if during tensioning of the system 10 minimal rotation of the stabilizing vertebrae outside the target region 24A is exhibited while derotation of the target region 24A is exhibited, the system 10 is determined to be more appropriately configured. If rotation of the stabilizing vertebrae outside of the target region 24A is apparent, it is determined to adjust the position of the stabilizing member 12 to better ensure that the reactive force 102 of the system 10 passes closer to the transverse centerline of rotation Y of the spinal column 24.

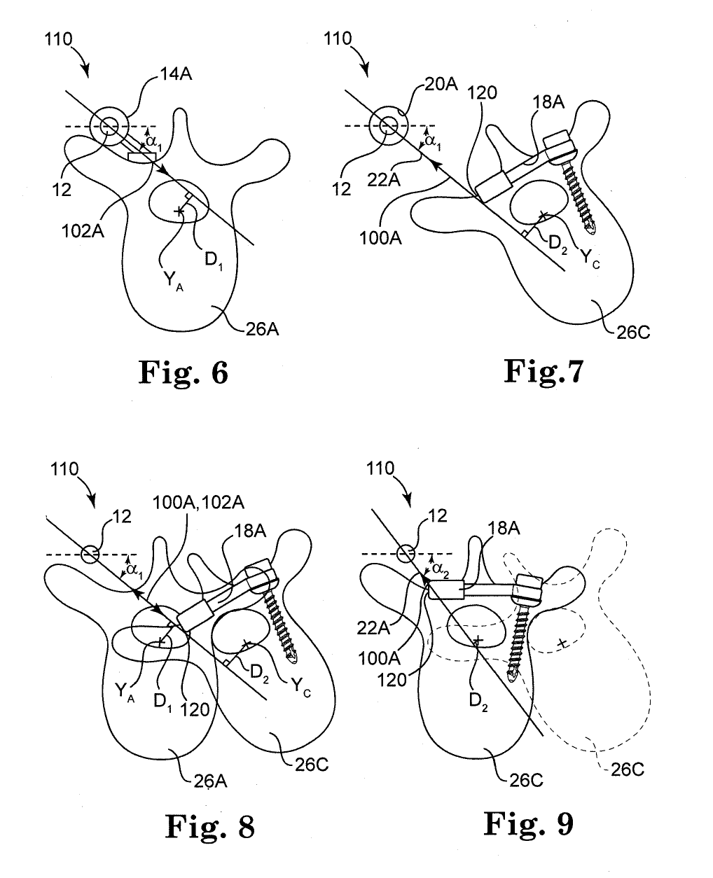

Methods and configurations for force balancing according to some other embodiments are described in greater detail below with reference to another system 110 shown in FIG. 5, where FIGS. 6-9 are also illustrative of force balancing concepts treated in association with the system 110. Various portions of the spinal column 24 and the system 110 are not shown in the views of FIGS. 6-9 for ease of illustration and understanding.

The system 110 is substantially similar to the system 10, but is simplified to include a single correction anchor 18 (the first correction anchor 18A), a single connector 22 (the first connector 22A), and a single tensioner 20 (the first tensioner 20A), the first correction anchor 18A being connected to the third vertebra 26C (e.g., an apical vertebra at the apex A of the curvature in the target region 24A). Similar force balancing concepts apply to other embodiment systems described herein, including the system 10. The system 110 is shown with four total stabilizing vertebrae 26 (the first and second stabilizing anchors 14A, 14B each being secured to two vertebrae 26), although greater or fewer stabilizing vertebrae 26 are employed as desired. For example, the first and second stabilizing anchors 14A, 14B are optionally each secured to a respective, single vertebra 26.

As with the system 10, the system 110 facilitates optimization of the relative direction, or force vector trajectory, of the push and pull forces—the corrective and reactive forces 100, 102—with respect to the transverse centerline of rotation Y in order to reduce rotational effect at the stabilizing vertebrae (e.g., the first and second vertebrae 26A, 26B), while encouraging derotation at the defect vertebrae (e.g., the third vertebra 26C).

In some embodiments, throughout the realignment or correction process (e.g., as the first tensioner 20A reduces the effective length of the first connector 22A), the force vector trajectories associated with the corrective and result forces 100, 102 change. For example, the corrective force 100 changes from a less steep to a more steep vector trajectory in some embodiments, allowing for increasing vertebral derotation in the target region 24A as the target region 24A translates laterally toward the stabilizing member 12 (e.g., as the target region 24A approaches a natural mid-line of the body). In some embodiments, the systems 110 is adapted such that corrective and resultant forces 100, 102 pass on opposite sides of the transverse centerline of rotation Y during one or more stages of correction and/or after correction.

FIG. 6 is a transverse plane representation of the system 110 at the first vertebra 26A indicative of a position of the stabilizing member 12 relative to the first stabilizing anchor 14A and the first vertebra 26A, according to some embodiments. FIG. 6 also shows a component of the reactive force 102 at the first vertebra 26A. Though not shown in FIG. 6, the stabilizing member 12 is substantially similarly positioned in the transverse plane relative to the second stabilizing anchor 14B and the second vertebra 26B, a similar component of the reactive force 102 being present at the second vertebra 26B, according to some embodiments.

As shown in FIG. 6, in some embodiments, the stabilizing member 12 is positioned at a pre-selected offset with respect to the first vertebra 26A such that tension in the first connector 22A causes a first component 102A of the reactive force 102 to pass the transverse center of rotation Y_{A} of the first vertebra 26A at a first perpendicular distance D1 at a first relative angle α₁. In some embodiments, the system 110 is configured to position the stabilizing member 12 such that the reactive force 102 passes substantially through the transverse center of rotation Y_{A} of the first vertebra 26A. In other words, D1 approaches or is substantially equal to zero in some embodiments in order to minimize rotational effects at the first vertebra 26A.

FIG. 7 is a transverse plane representation of the system 110 at the third vertebra 26C indicative of a position of the stabilizing member 12 relative to the first correction anchor 18A, the first connector 22A, and the third vertebra 26C, according to some embodiments. FIG. 7 shows the third vertebra 26C in a first, uncorrected position with the corrective force 100 being exerted on the third vertebra 26C at the first relative angle α₁.

As shown in FIG. 7, the first tensioner 20A is coupled to the stabilizing member 12 and the correction anchor 18A at a location along the stabilizing member 12 such that tension in the first connector 22A results in the first component 100A of the corrective force 100 (e.g., the first component 100A being equal to the corrective force 100) passing the transverse center of rotation Y_{C} of the third vertebra 26C at a second perpendicular distance D2.

FIG. 8 is an overlay of the representations of FIGS. 6 and 7 with the stabilizing member 12 position from FIG. 6 overlaid onto the stabilizing member 12 position from FIG. 7 and the component of the reactive force 102 from FIG. 6 overlaid onto the corrective force from FIG. 7. FIG. 9 is a transverse plane representation of the system 110 at the third vertebra 26C showing the third vertebra 26C rotated and translated to a second, corrected position.

As shown in the overlay of FIG. 8, the system 110 is configured to apply a pull effect, the first component 100A of the corrective force 100, at the second perpendicular distance D2 from the transverse center of rotation Y_{C} of the third vertebra 26C, resulting in a translational and derotational effect on the third vertebra 26C. In turn, the system 110 applies a resulting push effect, or stabilizing effect, the first component 102A of the reactive force 102, at the first perpendicular distance D1 from the transverse center of rotation Y_{A} of the first vertebra 26A.

In some embodiments, at initiation of or during correction of the target region 24A, and in particular the third vertebra 26C (FIG. 8), the relative difference between the first perpendicular distance D1 and the second perpendicular distance D2 is substantially greater than following correction of the target region 24A (FIG. 9), being greater in magnitude and/or extending in a generally opposite direction relative to the longitudinal centerline of rotation Y. As shown, the relative angle a first relative angle α₁ has changed to a second relative angle α₂. Thus, as alignment and correction continues to the more corrected, second position shown in FIG. 9, the first perpendicular distance D1 is substantially closer to D2, thereby facilitating maintenance of the correction (e.g., during movement such as twisting and/or bending) of the spinal column 24). In other words, there are reduced bending moments from the target region 24A (FIG. 5) on the system 110 from forces external to the system 110.

In terms of dimensional offsets, in some embodiments the stabilizing anchors 14A, 14B are configured to position the stabilizing member 12 at an offset from about 14 mm to about 30 mm lateral to the vertebral body mid-sagittal plane or the transverse center of rotation Y_{A} and from about 24 mm to about 32 mm posterior from the spinal canal 26 or the transverse center of rotation Y_{A} of the first vertebra 26A. The first correction anchor 18A is secured to the third vertebra 26C and is sized to define a vertebral correction point 120 for the third vertebra 26C. Additionally, in some embodiments, a length of the first correction anchor 18A is adjustable to select a location of the vertebral correction point 120. The vertebral correction point 120 corresponds to the location that is pulled upon from the stabilizing member 12 (e.g., at the terminal coupler 54A where the first connector 22A forms a polyaxial joint with the first correction anchor 18A).

In some embodiments, the vertebral correction point 120 is defined proximate (e.g., aligned or slightly lateral to) the concave pedicle entrance point and adjacent lamina of the third vertebra 26C. As previously described the vertebral correction point 120 is optionally on either side of the spinal column 24, but in some embodiments corresponds to the side of the spinal column 24 tending to exhibit a concave aspect of a defective curvature. In some embodiments, (e.g., as shown in the system 10) additional correction anchors 18 are similarly positioned on additional vertebrae 26 in the target region 24A. The stabilizing member 12 is also optionally positioned (e.g., by selecting a curvature of the stabilizing member 12 and location of the stabilizing anchors 18) to accomplish a desired force trajectory for the reactive force 102.

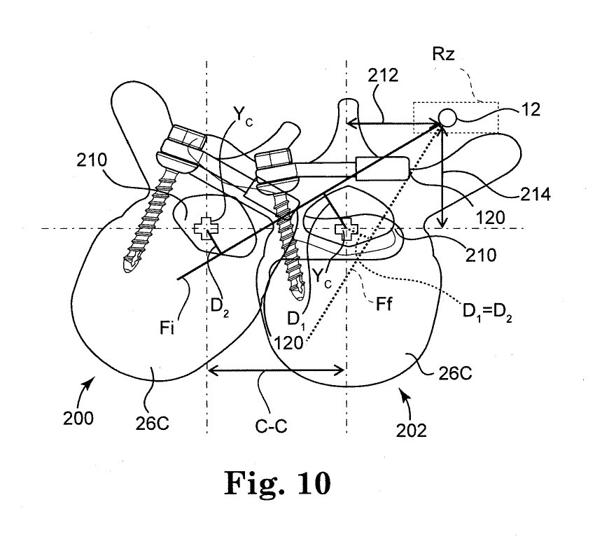

The following, non-limiting examples provide methodologies for calculating appropriate locations for the stabilizing member 12 with respect to the spinal column 24 and locations of the vertebral correction point 120 according to some embodiments. Reference is made to FIG. 10 in the following examples, which provides a transverse view of the third vertebra 26C in a first, uncorrected position 200 and a second, corrected position 202 and designates various features utilized for calculating the offset. In some embodiments, the second, corrected position 202 is treated interchangeably with a relative position of the stabilizing vertebrae (e.g., the first vertebra 26A), where the natural, desirable anterior-posterior curvature of the spinal column 24 is accounted for in such a model based upon the contour of the stabilizing member 12. FIG. 10 generally designates some acceptable placement zones Rz for the stabilizing member 12, where an initial force vector Fi changes to a final force vector Ff as the third vertebra 26C derotates and translates a center-to-center distance C-C from an initial position to a final position.

Various references include information relating to typical vertebral characteristic data, including, for example, Dennis R. Wenger et al., Biomechanics of Scoliosis Correction by Segmental Spinal Instrumentation, 7 SPINE 260 (1982); S. Rajasekaran et al., Eighteen-Level Analysis of Vertebral Rotation Following Harrington-Luque Instrumentation in Idiopathic Scoliosis, 76 J Bone Joint Surg Am. 104 (1994); Szabolcs Molnár et al., Ex Vivo and In Vitro Determination of the Axial Rotational Axis of the Human Thoracic Spine, 31 SPINE E984 (2006); James L. Berry et al., A Morphometric Study of Human Lumbar and Selected Thoracic Vertebrae, 12 SPINE 362 (1987); Ulf R. Liljenqvist et al., Analysis of Vertebral Morphology in Idiopathic Scoliosis with Use of Magnetic Resonance Imaging and Multiplanar Reconstruction, 84 J Bone Joint Surg Am. 359 (2002); AUGUSTUS A. WHITE III & MANOHAR M. PANJABI CLINICAL BIOMECHANCIS OF THE SPINE 28-29, Tbl. 1-5 (2d ed. 1990); Masaru Fujita et al., A Biomechanical Analysis of Sublaminar and Subtransverse Process Fixation Using Metal Wires and Polyethylene Cables, 31 SPINE 2202 (2006); Federico P. Girardi et al., Safety of Sublaminar Wires With Isola Instrumentation for the Treatment of Idiopathic Scoliosis, 25 SPINE 691 (2000), the entire contents of each of which are incorporated herein by reference.

In some embodiments, the third vertebra 26C is selected to be an apical vertebra of a thoracic, single curve deformity, where typical apical vertebrae for such a deformity includes T8 or T9 vertebra. The upper and lower stabilizing vertebrae, the first and second vertebrae 26A, 26B (FIG. 5), are selected as the T3-T4 vertebrae and the L1-L2 vertebrae, respectively, which are typical transitional vertebrae of a single, thoracic curve deformity. The transverse centerline of rotation Y (FIG. 5) for the target region 24A is selected at or near a mid-line, posterior edge of the vertebral bodies of the vertebrae 26 comprising the target region 24A, (which also corresponds to the transverse center of rotation Y_{C} of the third vertebra 26C). The medial-lateral, pedicle-to-pedicle dimension 210 for the T3-T9 vertebrae are selected to be about 25 mm to about 30 mm.

The transverse process width for the T3-T10 vertebrae is selected as being about 59 mm to about 60 mm, such that a maximum “X” dimension offset 212 for the stabilizing member 12 is estimated to be about 30 mm. The spinal canal depth for the T3-T10 vertebrae is estimated at approximately 16 mm, and based upon the instant axis of rotation (IAR), or transverse center of rotation Y_{C} being a few mm posterior to the vertebral body (e.g., of the third vertebra 26C), a minimum “Y” dimension offset 214 for the stabilizing member 12 from the transverse center of rotation Y_{C} of about 13 mm is selected. A midline vertebral depth from a position most anterior to most posterior at the T2-T7 vertebrae is selected as being about 64 mm. A vertebral body depth of the T2-T7 vertebrae is estimated at about 28 mm. Therefore, a maximum “Y” dimension offset 214, which is the maximum posterior offset of the stabilizing member 12 from the center of rotation Y_{C}, of about 36 mm is selected.

Widths of the T3-T10 vertebrae are estimated from about 25 to about 30 mm, for example being about 30 mm at the T10 vertebra. Thus, for a displacement of one vertebral body width at the third vertebrae 26C (e.g., the T8 or T9 vertebra), the third vertebrae 26C is translated an estimated distance of about 27 mm. The stabilizing member 12 is assumed to substantially rigidly remain at an essentially fixed distance from the stabilizing vertebrae (e.g., the first vertebra 26A), being located below the spinous processes and medial to the costovertebral joints.

A typical Cobb angle for operative candidates is estimated to be about 50 degrees to about 70 degrees, such that a translational distance from the first, uncorrected position 200 to the second, corrected position 202 measured between the stabilizing member 12 and the convex pedicle of the third vertebra 26C is about 12 mm to about 71 mm. Additionally, a derotation from the first, uncorrected position to the second, corrected position of about 25 degrees to about 30 degrees is selected, according to some embodiments.

In some embodiments, the contour of the stabilizing member 12 is selected to help ensure that the location of the stabilizing member 12 relative to the concave pedicles of the vertebrae 26 is approximately the same at the stabilizing vertebrae and the vertebrae 26 of the target region 24A once aligned in the coronal plane and translated to a fully corrected, natural position. In other words, the stabilizing member 12 is contoured such that the relative position of the stabilizing member 12 to the stabilizing vertebrae 26A, 26B (FIG. 5) is consistent with the relative position of the stabilizing member 12 to the third vertebra 26C upon translation to the second, corrected position.

Depending upon such factors as the initial, relative amount of rotation in the first, uncorrected position 200 and the center-to-center distance C-C, for example, the position of the stabilizing member 12 and/or the position of the vertebral correction point 120 is selected to encourage a desired rotational and lateral translation according to some embodiments. In particular, the system 110 allows positioning of the stabilizing member 12 and/or correction point 120 such that the lateral and derotational translational effects during correction are more readily controlled. In some embodiments, the length of the arm 50A of the first correction anchor 18A is increased to encourage more derotation at earlier stages of correction.

In some embodiments, lateral translation can be initiated first with derotational translation initiating at a later time during correction. In other embodiments, the reverse is optionally accomplished (derotation prior to lateral translation) or the relative amounts of derotational and lateral translation during various stages correction are pre-selected.

In the following examples, the distances D1 and D2 should be indicative of the moments applied at the apical vertebra and stabilizing vertebra and thus the relative amount of derotation accomplished for a defect of a particular severity and/or during a particular stage of correction. In particular, Tables 1-3 are demonstrative of operation of the system 110 in various configurations and under various loading conditions.

Table 1 indicates that according to some embodiments lateral translation will occur prior to derotation for a variety of locations of the stabilizing member 12, where there is an apical rotation of 20 degrees and center-to-center distance C-C of 28.5 mm. Table 2 indicates that, according to some embodiments, derotation should occur during defect correction for a variety of locations of the stabilizing member 12 at an apical rotation of about 30 degrees and a center-to-center distance C-C of 28.5 mm. Table 3 indicates, that, according to some embodiments as the Center-to-Center distance C-C decreases (e.g., during an intermediate phase of correction) the derotational moment at the apical vertebra, the third vertebra 26C (as indicated by the magnitude of D2) will be relatively high (e.g., in comparison to Table 1) while the rotational moment D1 on the stabilizing vertebra, the first vertebra 26A (as indicated by the magnitude of D1) will be relatively low (e.g., in comparison to Table 1) for various positions of the stabilizing member 12 where there is an apical rotation of 20 degrees.

Table 1 that follows illustrates a calculated offset of the stabilizing member 12 from the center of rotation Y_{C} of the third vertebra 26C and corresponding perpendicular distances D1 and D2 starting with an uncorrected rotation of about 20 degrees at the first, uncorrected position and ending at about 0 degrees for the second, corrected position.

TABLE 1
Inputs:

| | |
|---|---|
| Pedicle-to-Pedicle (approx. 25-30 mm in thoracic) | 28 |
| Anchor Arm Tip posterior to IAR (approx. 13 mm) | 13 |
| Apical Rotation (15-45 deg) | 20 |
| Center-to-Center Translation (approx 1+ vertebra, >27 mm) | 28.5 |

| | 1 | 2 | 3 | 4 | 5 | 6 | 7 | 8 | 9 |
|---|---|---|---|---|---|---|---|---|---|
| Position | | | | | | | | | |
| Stab. Member | 14.1 | 22.1 | 30.0 | 14.1 | 24.3 | 30.0 | 14.1 | 22.1 | 30.0 |
| “X” distance (approx 12-30 mm; | | | | | | | | | |
| existing joint @ 14 | | | | | | | | | |
| or 24.3) | | | | | | | | | |
| Stab. Member | 24.0 | 24.0 | 24.0 | 28.0 | 28.0 | 28.0 | 30.0 | 30.0 | 30.0 |
| “Y” distance (approx 24-32 mm; | | | | | | | | | |
| existing joint @ 30 | | | | | | | | | |
| or 28) | | | | | | | | | |
| Initial Position | | | | | | | | | |
| Vector | | | | | | | | | |
| Anchor tip | −10.9 | −10.9 | −10.9 | −10.9 | −10.9 | −10.9 | −10.9 | −10.9 | −10.9 |
| position X′ | | | | | | | | | |
| Anchor tip | 7.4 | 7.4 | 7.4 | 7.4 | 7.4 | 7.4 | 7.4 | 7.4 | 7.4 |
| position Y′ | | | | | | | | | |
| Vector Angle | 33.5 | 26.7 | 22.1 | 39.5 | 30.3 | 26.7 | 42.1 | 34.4 | 28.9 |
| (deg) | | | | | | | | | |
| D1 (end vertebra | −12.2 | −11.5 | −11.0 | −12.7 | −11.9 | −11.5 | −12.8 | −12.3 | −11.8 |
| moment arm, | | | | | | | | | |
| mm) | | | | | | | | | |
| D2 (apical | 5.3 | 2.5 | −0.7 | 6.6 | 4.2 | 2.5 | 7.0 | 5.6 | 3.6 |
| vertebra moment | | | | | | | | | |
| arm, mm) | | | | | | | | | |
| Final Position Vector | | | | | | | | | |
| Vector Angle (deg) | 89 | 54 | 35 | 90 | 56 | 43 | 90 | 65 | 47 |
| D1 = D2 (end vertebra moment | 13.9 | 3.6 | −2.8 | 13.9 | 4.2 | 0.1 | 13.9 | 7.1 | 1.3 |
| arm, mm) | | | | | | | | | |

Table 2 that follows illustrates a calculated offset of the stabilizing member 12 from the center of rotation Y_{C} of the third vertebra 26C and corresponding perpendicular distances D1 and D2 starting with an uncorrected rotation of about 30 degrees at the first, uncorrected position with a center-to-center translation C-C of about 28.5 mm and ending at about 0 degrees for the second, corrected position.

TABLE 2
Inputs:

| | |
|---|---|
| Pedicle-to-Pedicle (approx. 25-30 mm in thoracic) | 28 |
| Anchor Arm Tip posterior to IAR (approx. 13 mm) | 13 |
| Apical Rotation (15-45 deg, figure is @ approx 30 deg) | 30 |
| Center-to-Center Translation (approx 1+ vertebra, >27 mm) | 28.5 |

| | 1 | 2 | 3 | 4 | 5 | 6 | 7 | 8 | 9 |
|---|---|---|---|---|---|---|---|---|---|
| Position | | | | | | | | | |
| Stab. Member | 14.1 | 22.1 | 30.0 | 14.1 | 24.3 | 30.0 | 14.1 | 22.1 | 30.0 |
| “X” distance (approx 12-30 mm; | | | | | | | | | |
| existing joint @ 14 | | | | | | | | | |
| or 24.3) | | | | | | | | | |
| Stab. Member | 24.0 | 24.0 | 24.0 | 28.0 | 28.0 | 28.0 | 30.0 | 30.0 | 30.0 |
| “Y” distance (approx 24-32 mm; | | | | | | | | | |
| existing joint @ 30 | | | | | | | | | |
| or 28) | | | | | | | | | |
| Initial Position | | | | | | | | | |
| Vector | | | | | | | | | |
| Anchor tip | −9.9 | −9.9 | −9.9 | −9.9 | −9.9 | −9.9 | −9.9 | −9.9 | −9.9 |
| position X′ | | | | | | | | | |
| Anchor tip | 4.3 | 4.3 | 4.3 | 4.3 | 4.3 | 4.3 | 4.3 | 4.3 | 4.3 |
| position Y′ | | | | | | | | | |
| Vector Angle | 39.5 | 31.7 | 26.3 | 44.7 | 34.8 | 30.8 | 47.0 | 38.9 | 32.8 |
| (deg) | | | | | | | | | |
| D1 (end vertebra | −9.6 | −8.8 | −8.2 | −10.0 | −9.1 | −8.7 | −10.1 | −9.5 | −8.9 |
| moment arm, | | | | | | | | | |
| mm) | | | | | | | | | |
| D2 (apical | 10.4 | 10.0 | 9.0 | 10.2 | 10.3 | 9.9 | 10.0 | 10.4 | 10.1 |
| vertebra moment | | | | | | | | | |
| arm, mm) | | | | | | | | | |
| Final Position Vector | | | | | | | | | |
| Vector Angle (deg) | 89 | 54 | 35 | 90 | 56 | 43 | 90 | 65 | 47 |
| D1 = D2 (end vertebra moment | 13.9 | 3.6 | −2.8 | 13.9 | 4.2 | 0.1 | 13.9 | 7.1 | 1.3 |
| arm, mm) | | | | | | | | | |

Table 3 that follows illustrates a calculated offset of the stabilizing member 12 from the center of rotation Y_{C} of the third vertebra 26C and corresponding perpendicular distances D1 and D2 starting with an uncorrected rotation of about 20 degrees at the first, uncorrected position with a center-to-center translation C-C of about 14.25 mm and ending at about 0 degrees for the second, corrected position. As previously referenced, Table 3 is indicative of the anticipated derotational effect of corrective forces (e.g., at an intermediate stage of correction) according to some embodiments.

TABLE 3
Inputs:

| | |
|---|---|
| Pedicle-to-Pedicle (approx. 25-30 mm in thoracic) | 28 |
| Anchor Arm Tip posterior to IAR (approx. 13 mm) | 13 |
| Apical Rotation (15-45 deg, figure is @ approx 30 deg) | 20 |
| Center-to-Center Translation (approx 1+ vertebra, >27 mm) | 14.25 |

| | 1 | 2 | 3 | 4 | 5 | 6 | 7 | 8 | 9 |
|---|---|---|---|---|---|---|---|---|---|
| Position | | | | | | | | | |
| Stab. Member | 14.1 | 22.1 | 30.0 | 14.1 | 24.3 | 30.0 | 14.1 | 22.1 | 30.0 |
| “X” distance (approx 12-30 mm; | | | | | | | | | |
| existing joint @ 14 | | | | | | | | | |
| or 24.3) | | | | | | | | | |
| Stab. Member | 24.0 | 24.0 | 24.0 | 28.0 | 28.0 | 28.0 | 30.0 | 30.0 | 30.0 |
| “Y” distance (approx 24-32 mm; | | | | | | | | | |
| existing joint @ 30 | | | | | | | | | |
| or 28) | | | | | | | | | |
| Initial Position | | | | | | | | | |
| Vector | | | | | | | | | |
| Anchor tip | 3.4 | 3.4 | 3.4 | 3.4 | 3.4 | 3.4 | 3.4 | 3.4 | 3.4 |
| position X′ | | | | | | | | | |
| Anchor tip | 7.4 | 7.4 | 7.4 | 7.4 | 7.4 | 7.4 | 7.4 | 7.4 | 7.4 |
| position Y′ | | | | | | | | | |
| Vector Angle | 57.0 | 41.6 | 31.9 | 62.4 | 44.5 | 37.7 | 64.5 | 50.4 | 40.3 |
| (deg) | | | | | | | | | |
| D1 (end | −1.2 | −3.3 | −4.5 | −0.5 | −3.0 | −3.8 | −0.2 | −2.2 | −3.5 |
| vertebra | | | | | | | | | |
| moment arm, | | | | | | | | | |
| mm) | | | | | | | | | |
| D2 (apical | 7.0 | 6.9 | 4.8 | 6.4 | 7.2 | 6.3 | 6.0 | 7.3 | 6.7 |
| vertebra | | | | | | | | | |
| moment arm, | | | | | | | | | |
| mm) | | | | | | | | | |
| Final Position Vector | | | | | | | | | |
| Vector Angle (deg) | 89 | 54 | 35 | 90 | 56 | 43 | 90 | 65 | 47 |
| D1 = D2 (end vertebra | 13.9 | 3.6 | −2.8 | 13.9 | 4.2 | 0.1 | 13.9 | 7.1 | 1.3 |
| moment arm, mm) | | | | | | | | | |

The foregoing exemplary methodology is meant to be illustrative in nature, other techniques being contemplated. Moreover, and as previously mentioned, additional components (e.g., additional correction anchors 18, tensioners 20, and connectors 22) are useful in accomplishing balanced application of reactive forces having reduced rotational effects on the stabilizing vertebrae. For example, according to some embodiments, the system 10 is adapted to direct the components of the reactive force 102 relatively closer to the longitudinal centerline of rotation Y in comparison to the components of the corrective force 100 to achieve a desired derotation effect on the target region 24A while reducing unwanted twisting, or rotation, of the stabilizing vertebrae such as the first and second vertebrae 26A, 26B.

In view of the foregoing, various embodiment systems described herein utilize a corrective force applied in a target region (e.g., at or near an apex of a spinal deformity) for inducing spinal realignment (translation and derotation) that resists transferring inducing a compensating deformity (e.g., complementary rotation) into stabilizing vertebral bodies (e.g., vertebrae near the cephalad and caudal ends of a defect region of a spinal deformity) to which stabilizing anchors of the system are secured. Controlled realignment of a deformity (translation and derotation) is optionally accomplished with minimal attachment points to stabilizing vertebrae and corrective vertebra(e) (e.g., as few as three total) with reduced potential for inducing a compensatory curve outside the target region of the spinal column being corrected.

Various modifications and additions can be made to the exemplary embodiments discussed without departing from the scope of the present invention. While the embodiments described above refer to particular features, the scope of this invention also includes embodiments having different combinations of features and embodiments that do not include all of the described features. Accordingly, the scope of the present invention is intended to embrace all such alternatives, modifications, and variations as fall within the scope of the claims, together with all equivalents thereof.

What is claimed is:

1. A system for correction of a spinal column having a target region exhibiting a spinal deformity, the spinal column including a first stabilizing vertebra proximate a first end of the region of spinal deformity, a second stabilizing vertebra proximate a second end of the region of spinal deformity, and a correction vertebra between the first and second stabilizing vertebrae where the spinal column defines a transverse centerline of rotation and the stabilizing vertebrae and the correction vertebra each have a transverse center of rotation along the transverse centerline of rotation, the system comprising:
a stabilizing member adapted to extend between the first stabilizing vertebra and the second stabilizing vertebra;
a first stabilizing anchor adapted to locate the stabilizing member with respect to the first stabilizing vertebra;
a second stabilizing anchor adapted to locate the stabilizing member with respect to the second stabilizing vertebra;
a correction anchor adapted to be secured to the correction vertebra; and
a connector secured between the stabilizing member and the correction anchor, wherein the stabilizing member, the first and second stabilizing anchors, and the correction anchor are configured such that upon tensioning the connector a corrective force is exerted on the correction vertebra and a reactive force is exerted on the first and second stabilizing vertebrae, the reactive force passing on a first side of the transverse centerline of rotation and the corrective force passing one of substantially through the transverse centerline of rotation and on a second side of the transverse centerline of rotation that is opposite the first side of the transverse centerline of rotation.

2. The system of claim 1, wherein the corrective force passes relatively further from the transverse center of rotation of the correction vertebra than the reactive force passes from the transverse centers of rotation of each of the first and second stabilizing vertebrae.

3. The system of claim 1, wherein the connector allows for angular changes in the corrective force with respect to the stabilizing member.

4. The connector of claim 1, wherein the connector is substantially flexible.

5. The system of claim 1, further comprising a tensioner adjustably coupling the connector to the stabilizing member.

6. The system of claim 1, further comprising a plurality of correction anchors secured to the target region and a plurality of connectors secured between the stabilizing member and the plurality of connectors, wherein the corrective force comprises a summation of the tension in the connectors.

7. The system of claim 1, wherein the stabilizing member is offset from the transverse center of rotation of the first stabilizing vertebra in the posterior direction from about 24 mm to about 32 mm.

8. The system of claim 1, wherein the stabilizing member is offset from the transverse center of rotation of the first stabilizing vertebra in lateral direction that is perpendicular to the posterior direction from about 14 mm to about 30 mm.

9. A method of correcting a spine tending to exhibit a defective curvature in a target region of the spine, where the spine has a longitudinal centerline of rotation and is defined by a plurality of vertebrae, the method comprising:
securing a first stabilizing anchor to a first stabilizing vertebra residing adjacent a first end of the target region, the first stabilizing vertebra having a transverse center of rotation and a rotational orientation about the longitudinal centerline of rotation of the spine;
securing a second stabilizing anchor to a second stabilizing vertebra residing adjacent a second end of the target region that is opposite the first end of the target region;
attaching an elongate stabilizing member to the first and second stabilizing anchors;
securing one or more correction anchors to one or more defect vertebrae forming the target region, the defect vertebrae tending to exhibit a rotational misalignment with the rotational orientation of the first stabilizing vertebra;
tensioning the one or more correction anchors to the stabilizing member to impose a corrective force on the one or more defect vertebrae such that the one or more defect vertebrae are substantially aligned to the rotational orientation of the first stabilizing vertebra, where the corrective force is at a first transverse offset from the longitudinal centerline of rotation, the corrective force having a complementary reactive force on the spine at the first and second stabilizing vertebrae;
controlling a rotational effect on the first stabilizing vertebra from the reactive force by directing the reactive force toward the transverse center of rotation of the first stabilizing vertebra.

10. The method of claim 9, wherein the corrective force extends at a transverse angle from the stabilizing member and acts to rotate the one or more defect vertebrae and laterally translate the one or more defect vertebrae into rotational and sagittal alignment with the first and second stabilizing vertebrae.

11. The method of claim 10, wherein the transverse angle moves toward a posterior-anterior axis as the one or more defect vertebra rotate and laterally translate into rotational and sagittal alignment with the first and second stabilizing vertebrae.

12. The method of claim 9, wherein the first stabilizing anchor, the second stabilizing anchor, and the stabilizing member are positioned on a side of the spine corresponding to a concave aspect of the defective curvature.

13. The method of claim 9, wherein each of the first and second stabilizing vertebrae has a transverse center of rotation on the transverse centerline of rotation of the spine, wherein the reactive force includes a first component at the first stabilizing vertebra and a second component at the second stabilizing vertebra, and further wherein controlling the rotational effect on the first and second stabilizing vertebrae from the reactive force includes directing the first and second components toward the transverse centers of rotation of the first and second stabilizing vertebrae, respectively.

14. The method of claim 9, wherein the target region has an apical vertebra characterizing the defective curvature, the method further comprising selecting a transverse offset of the stabilizing member from the spinal column at the first and second stabilizing vertebrae using an apical vertebral rotation angle and apical vertebral translation from the sagittal plane.

15. The method of claim 9, wherein the target region has an apical vertebra characterizing the defective curvature, the method further comprising selecting a transverse offset of the stabilizing member from the spine by selecting a contour of the stabilizing member.

16. The method of claim 9, wherein a magnitude of the corrective force is a summation of the tension in the one or more connectors.

17. The method of claim 9, further comprising positioning the stabilizing member at an offset from the transverse center of rotation of the first stabilizing vertebra in the posterior direction from about 24 mm to about 32 mm.

18. The method of claim 9, further comprising positioning the stabilizing member at an offset from the transverse center of rotation of the first stabilizing vertebra in lateral direction that is perpendicular to the posterior direction from about 14 mm to about 30 mm.

19. A method of derotating and laterally translating a targeted deformed area of a spine, the spine including stabilizing vertebrae and target vertebrae along the targeted deformed area, the method comprising:
securing alignment member anchors to the stabilizing vertebrae and inserting a stabilizing member between stabilizing vertebrae to establish a line of reference from which to adjust a position of the target vertebrae by pulling on the target vertebrae from the line of reference;
establishing at least one vertebral correction point along the targeted deformed area by securing at least one correction anchor to at least one target vertebra, each of the correction points being disposed at a lateral offset from a transverse center of rotation of a corresponding target vertebra; and
pulling against the line of reference towards the alignment member to impart translation and derotation of target vertebrae and maintain lateral translation and derotation of the targeted deformed area of the spine.

20. The method of claim 19, further comprising selecting a transverse position of the stabilizing member relative to the spine to minimize moments on the stabilizing vertebrae during pulling against the line of reference.

21. A method of correcting a spine tending to exhibit a defective curvature in a target region of the spine, where the spine has a longitudinal centerline of rotation and includes a plurality of vertebrae, the method comprising:
securing a first stabilizing anchor to a first stabilizing vertebra residing adjacent a first end of the target region, the first stabilizing vertebra having a stabilizing transverse center of rotation and a rotational orientation about the longitudinal centerline of rotation of the spine;
securing a second stabilizing anchor to a second stabilizing vertebra residing adjacent a second end of the target region that is opposite the first end of the target region;
attaching an elongate stabilizing member to the first and second stabilizing anchors, such that the stabilizing member extends across the target region;
securing at least one correction anchor to at least one deformed vertebra forming the target region, the deformed vertebra having a corrective transverse center of rotation and tending to exhibit a rotational misalignment with respect to the rotational orientation of the first stabilizing vertebra;
tensioning the correction anchor to the stabilizing member to impose a corrective force on the one or more defect vertebrae;
wherein at least one of the stabilizing anchors and the correction anchor is configured such that upon tensioning, at a corrected position, the correction anchor imparts a corrective force upon the deformed vertebra at a first distance from the corrective transverse center of rotation and a complementary reactive force on the first and second stabilizing vertebrae at a second distance from the stabilizing transverse center of rotation, such that the first distance is greater than or equal to the second distance.
